(19) United States
(12) Patent Application Publication
(10) Pub. No.: US 2023/0210489 A1
(43) Pub. Date: Jul. 6, 2023

(54) EXTENDED AUSCULTATION DEVICE

(71) Applicant: IDTM GmbH, Recklinghausen (DE)

(72) Inventors: Axel BOESE, Magdeburg (DE); Michael FRIEBE, Recklinghausen (DE); Rutuja SALVI, Magdeburg (DE); Thomas SÜHN, Magdeburg (DE); Moritz SPILLER, Magdeburg (DE); Stefan HELLWIG, Hemer (DE); Alfredo ILLANES MANRIQUEZ, Magdeburg (DE)

(73) Assignee: IDTM GmbH, Recklinghausen (DE)

(21) Appl. No.: 17/927,712

(22) PCT Filed: May 26, 2021
(86) PCT No.: PCT/EP2021/064069

§ 371 Date: Nov. 24, 2022

(30) Foreign Application Priority Data
May 26, 2020 (DE) 10 2020 114 070.3

(51) Int. Cl. A61B 7/04; A61B 7/00; A61B 5/00

(52) U.S. Cl. CPC A61B 7/04; A61B 7/003; A61B 5/726; A61B 5/7257

(57) ABSTRACT

The invention relates to an auscultation device (1) for capturing and evaluating body sounds, having an auscultation element (2) having a housing (4, 4a) and arranged therein: an audio system (5), a power supply (6), a computing unit (7), means for data transmission (7a) and a control interface (8), and having a data processing system having a user interface (10), means for data transmission (14a), a memory (13) and a computing unit (14), wherein the audio system (5) comprises at least one microphone unit (5a, 5b) for capturing the body's own sounds and this at least one microphone unit (5a, 5b) comprises a microphone (i) and a connection element (ii) between microphone (i) and housing (4, 4a).

The invention relates to an auscultation device for recording and analyzing the body's own sounds in the region of the neck, more particularly in the region of the carotid branch.

According to information in the WHO mortality database, cerebrovascular diseases (CVD) are responsible for around one million deaths per year in Europe alone. Around 85% of all CVD deaths relate to an ischemic stroke (IS).

A cerebrovascular ischemic event occurs when an atherosclerotic stenosis or a sudden closure of a large vessel prevents the arterial blood supply to parts of the brain. Such an event can either cause a merely temporary ischemic attack or permanently lead to an ischemic stroke.

In particular the atherosclerosis of the carotid artery (Arteria carotis communis, carotid for short) is a frequent cause of such events. The two carotids are located on the left and right side of the neck and supply head and brain with oxygen-containing blood. A dissection or an atherosclerosis can lead to a carotid stenosis (CS).

The diagnosis and monitoring of atherosclerotic conditions of the carotid are currently based on non-invasive imaging technologies such as ultrasound (US), computed tomography angiography (CT) and magnetic resonance angiography (MRT), the latter being indicated in symptomatic patients and high-risk patients or if US is not possible. Due to its inherent risk of complications, invasive catheter angiography is reserved for patients who are contraindicated for a non-invasive imaging or whose results are not unambiguous.

While the current diagnostic methods already frequently offer an exact assessment of the severity of the stenosis and an appraisal of anatomical details, they also have a number of disadvantages. To carry out most known methods, for example, it is necessary to acquire expensive and bulky equipment, which furthermore can only be operated by specially trained and experienced physicians and employees in a clinical institution. In addition, some imaging methods require the use of ionizing radiation or contrast media, some of which have considerable side effects.

In contrast, auscultation, i.e. listening to internal sounds of the body using a stethoscope, is a low-cost and conventional diagnostic method in clinical practice. The method is widespread, but requires extensive clinical experience and a well-developed sense of hearing. Ultimately, however, the appraisal of audible body sounds remains largely subjective.

The screening of carotid sounds for thrombi, i.e. audible vessel sounds in conjunction with a turbulent blood flow in the vessel, is recommended as an important part of routine physical examinations of patients with risk factors for vascular diseases. Suspicious sounds can be connected with vascular pathologies or changes in the vascular status and are in the range between 20 and 500 Hz.

Most studies and common guidelines for treating atherosclerotic vascular diseases agree that corresponding carotid sounds are suitable as an indicator for CS and thus as a justification for the use of further diagnostic imaging methods.

In contrast to the previous assessment of the presence of carotid sounds using relatively simple means such as a conventional stethoscope, a digital stethoscope, phonocardiography or seism cardiography, the most recent developments in sensor technology, signal processing and computer-assisted learning (artificial intelligence, AI) permit a more specific assessment of such sounds or signals or data and the pathological sound properties corresponding to same.

Thus corresponding data processing processes can for example be used to obtain, from the captured data, information (for example the presence of certain frequencies and their proportion, energy, amplitude etc.) that even a proficient person could not identify even with the aid of a digital amplification of the signal, and thus could not use for a diagnosis.

With an enhanced auscultation device which uses the latest sensor technologies, signal processing and AI, the vascular status could be used as a cost-effective and simple alternative to known diagnostic imaging methods, for example by means of a periodically conducted auscultation at the point-of-care location or at home.

Furthermore, such an enhanced auscultation device would permit the implementation of personalized management strategies as well as a continuous monitoring of vascular diseases which are suspected, developing, advanced or being treated.

Not least, it is conceivable that in addition to the sounds of the carotids, other body sounds can be recorded and evaluated with the aid of such an auscultation device for the diagnosis of pathological conditions.

The object of the invention is therefore that of providing an auscultation device which can capture and evaluate sounds not only of the carotids, but also permits the capture of other body sounds and the evaluation thereof acutely or over a longer period.

In addition, the auscultation device should preferably be designed so compactly that it can be worn by a patient optionally also over a longer time on the body part for capturing—such as more particularly the neck to record the carotid sounds or the chest to record lung and heart sounds, wherein every other body part is also possible—without this leading to unpleasant restrictions.

Furthermore, the auscultation device should permit an evaluation of the relevant body sounds which is objective and largely uninfluenced by interference sounds.

This object is achieved by an invention having the features of claim 1. Advantageous embodiments are the subject-matter of the dependent claims. It is noted that the features individually listed in the claims can be combined with one another in arbitrary and technologically meaningful manner and thus show further embodiments of the invention.

A computer-assisted auscultation device (CAAS) comprises a compact, hand-held auscultation element and an electric data processing system (EDP) connected thereto.

Advantageous on the hand-held auscultation element is the comfortable and reproducible capture of vessel sounds for the subsequent analysis. The auscultation element must be easy to handle so that it can also be used by non-expert users in everyday use. Thus the data capture using the auscultation element according to the invention should as a rule be of comparable complexity and duration as an indirect blood pressure measurement using an ultra-modern blood pressure cuff or the measurement of the oxygen saturation of the blood with the aid of an infrared sensor on the finger.

The auscultation element comprises a compact housing in which mainly an audio system for data capture, namely for recording sounds, and means for data transmission, a computing unit as control system, a control interface and a power supply are arranged. The presence of further elements such as circuit boards and corresponding electronic elements are a matter of course for a person skilled in the art.

The housing has at least one lateral face which is suitable for resting against the body surface of the patient. The housing parts which come into contact with the patient can be provided in a sterilizable manner, optionally together with at least part of the audio system.

Alternatively, these parts of the housing and/or of the audio system can be provided to be exchangeable. This permits for example the use of different microphones or supports, as a possible disposable item, to comply with applicable hygiene standards. Alternatively, these parts can also be physically separate from the other elements and be connected to same by a wired or wireless connection.

The audio system comprises at least one microphone. Preferably, the audio system comprises a plurality of microphones, which are provided for recording different frequencies and/or different dynamic ranges and/or with different sensitivities. Further elements can be associated with the audio system, for example parts of the computing unit or parts of the circuit board plus electronics needed for the arrangement and internal linking of the microphones. For the above-mentioned reasons, these elements can likewise be provided to be exchangeable.

The microphones are arranged in the housing such that they can best capture the sounds which they are provided to record. For this, the microphones are provided for capturing the body's own sounds preferably on the side of the housing that is to rest against the patient's body.

Preferably, the housing has at least partial openings at these points. Between the microphone and the respective housing region, hollow-cylindrical connection elements can be provided which foster the sound transmission from the body surface to the microphone. The hollow-cylindrical connection elements are preferably funnel-shaped and widen from the microphone to the housing wall.

The openings in the housing wall can optionally be covered by a membrane. The membrane, together with the microphone, the connection element and the housing wall, thus forms a closed space. This makes sense in order to prevent an energy loss of the sound waves, in particular if air-coupled microphones are used.

The microphones for capturing the body's own sounds are preferably shielded against extraneous sounds.

It is conceivable that at least one of the microphones is more particularly provided to capture exogenous sounds, which can be used for example in the subsequent data processing for a signal correction of the body's own sounds. Microphones provided to this end can also be provided on sides of the housing which do not rest against the patient's body. Openings in the housing wall can correspondingly be provided for these microphones as well, which openings are optionally equipped with a funnel and a membrane. Such microphones, which are more particularly provided for capturing exogenous sounds, can additionally or alternatively also be provided for recording the external portion of the body's own sounds, for example for the optionally merely supplementary recording of coughing sounds.

The microphone unit(s) for recording exogenous sounds can also be physically separate from the other elements of the auscultation element and connected to the same by a wired or wireless connection.

The body's own sounds are understood to be those sounds that result from the functions of the body per se. These include for example breathing sounds such as sounds of the trachea and bronchi, swallowing sounds and sounds of the esophagus, digestion sounds or sounds of the heartbeat and blood flow, to name just a few at this juncture.

Exogenous sounds, on the other hand, are understood to be those sounds which arise mainly outside of the body and are captured by the microphones either directly or through the sound transmission of the body. Such sounds include all external sounds such as ambient sounds or sounds that result from the interaction of the body with the environment.

The terms sound, signal and data are in some cases used as equivalents in the description, because for the inventive concept, it is as a rule irrelevant to define whether each specific case relates to the physical variable (the sound in the form of a sound wave) or the measurement signal (for example an electrical alternating voltage) or the (digital) data. Ultimately the auscultation device or the auscultation element converts a physical variable into digital data. As a rule, however, there is at least differentiation between sounds as a physical variable and digital data.

For the respective use, two types of microphone are in particular possible: direct contact microphones and air-coupled microphones.

Conventional contact microphones use the piezoelectric effect to convert mechanical energy into an electric signal. This has two main disadvantages for the capture of body sounds via the skin. First, the microphone must be in fixed contact with the skin, wherein the mechanical load on the skin from the sound head must simultaneously be minimal so as not to adversely affect the sound transfer. However, sufficient contact is difficult to achieve not least because of the unevenness of the body surface.

Air-coupled microphones capture the vibrations of the skin via the air. Compared to contact microphones, this provides for a lower load on the skin, but such microphones also capture unwanted ambient noises more quickly. Furthermore, the use of air-coupled microphones required a fixed reference frame which ensures a defined transmission distance and a defined air volume between skin and microphone.

In addition to microphones as typical acoustic sensors or sound converters, further sensors can be provided as part of the auscultation device, which sensors provide, for example, information about body temperature, the conductivity of the skin etc. The further sensors, for example heat sensors or conductivity sensors, can be accommodated in the housing of the auscultation element and be exchangeable—optionally with parts of the housing as described above. However, the further sensors can also be provided separate from the auscultation element at other places on the body. The data of these sensors can flow into the subsequent data analysis.

For releasable fastening on the body of the patient, preferably on the skin surface on the neck at the level of the carotid branch, the auscultation device optionally comprises at least one fastening element, for example in the form of a corresponding strap system, an adhesive strip, a plaster etc. By means thereof, the auscultation element can be fixed at a place favorable for data capture, thereby minimizing the risk of slipping, as compared to a manual holding, during data capture. The respective fastening element can comprise variations for different body measurements, for example a butterfly design for children, youths or adults and for large or very large people.

The data obtained via the sensors or microphones can be transmitted from the auscultation element in a wireless or wired manner to external apparatuses, for example the data capture system of the auscultation device.

The auscultation element comprises a computing unit, which is mainly provided for controlling the individual elements of the auscultation element. This includes for example the data transmission or the operation via the control interface.

The control interface preferably has a simple structure and is used above all to operate basic functions of the auscultation element, such as switching the auscultation element on and off, and starting or ending a recording. Simple keys for example can be provided for this.

The control interface can also comprise more particularly acoustic and/or optical signal generators, which provide information for example about the respective operating state of the auscultation element or the quality of the data capture.

The auscultation device according to the invention further comprises, in addition to the auscultation element, a data processing system having an analysis and diagnostic software.

The data processing system receives the data obtained by means of the auscultation element. The data transmission between auscultation element and data processing system can be wireless or wired. The data processing system in turn can be connected in a wireless or wired manner to further data processing systems or a corresponding network.

As wireless connections, all known standards can be used which ensure sufficient data transmission, such as for example Bluetooth, Wi-Fi, infrared etc. Corresponding means of data transmission are provided both as part of the auscultation element and as part of the data processing system. The means for data transmission are preferably provided closely connected to the respective computing units and optionally on the board of the computing unit.

It is conceivable that in certain embodiments, a data processing system is directly integrated in the auscultation element. Embodiments are also conceivable in which the data processing is carried out by for example a smart phone or a tablet as possible data processing systems.

The data processing system according to the invention is provided for visualization of the data captured by means of the auscultation element and for an analysis and diagnosis method using the data captured and transmitted by means of the auscultation element.

In a first step of the analysis and diagnosis method of pathological states, a recording of the body's own sounds with as little interference as possible should be ensured.

For this, an initial data recording is carried out using the auscultation element and must be successfully completed.

In the initial data recording, the audio signals of at least two heart impulses, a swallowing sound, the blood flow of the carotid, a cough and a breath should preferably be successfully captured.

The initial data recording is deemed to be successful if, in a comparison, the data to be captured meet previously defined parameters which indicate a successful data recording. Such parameters result in particular from known successful data recordings. Such a comparison can be carried out for example by comparing the initial data recording with a stored reference signal. The success or quality of the initial data recording can then be displayed using an optical element. A green light for example indicates that the data recording meets the requirements, a red light that the data recording does not meet the requirements and should be repeated. The first analysis therefore serves primarily to assess the quality of the recording.

The further information which can be captured during a possible enhanced initial data recording includes sneezing, throat-clearing and information about heart valve sounds, a more in-depth characterizing of the carotid and possible stenoses as well as other audio and flow information which is not directly attributable.

The recording of such further sounds can help better assess the recording quality. The recording of such further sounds can also improve the subsequent data evaluation and diagnosis of pathological states.

The data obtained in the initial data recording, hereinafter also referred to as the initial dataset, are, after an optional prefiltering, compared with known data and thus the recording quality of said initial dataset is assessed. The comparison can be carried out by a person skilled in the art for example using the visualization of the audio data or directly by the data processing system with the aid of certain algorithms.

The initial data recording lasts between 1 and 100 seconds. Preferably, a duration of 10 to 60 seconds is intended for the entire data recording, wherein individual partial recordings (for example of the heart sounds) can be in the range of 1 to 10 seconds.

If the data quality achieved by the initial recording is deemed to be sufficient based on the data comparison, this is confirmed by the auscultation system based on a signal, wherein the type of signal can be freely selected and can comprise for example an optical and/or acoustic and/or tactile signal. The signal can for example be output via the control interface of the auscultation element. Conceivable here would be a green light for a recording of sufficient quality and a red light for a recording of insufficient quality. Many other signal variants are conceivable.

If sufficient data quality could not be achieved with the initial data capture, the capture must be repeated until sufficient data quality has been achieved. As a rule, the auscultation element must be adjusted to a new position for this.

The above-described steps in conjunction with the initial recording can also be understood as a calibration or initialization of the auscultation device or auscultation element. The calibration ensures that the basic requirements for recordings of a quality sufficient for further processing have been met.

The recording of the data relevant for the evaluation or diagnosis, herein also referred to as the main dataset, is carried out only after successful calibration.

The duration of the recording of the main dataset depends more particularly on the type of diagnosis to be made. The duration is also not essential to the invention. The frequency, i.e. the repetition of the recordings, also depends on the type of diagnosis to be made and is not essential to the invention.

Thus in one case, a successful and reliable diagnosis of pathological states may already be possible after a one-time data capture over just a few seconds, and in another case, a reliable diagnosis is only possible after a plurality of data captures over a longer period. Ultimately, different intervals and recording lengths are possible here. A corresponding history can also for example be documented over years and be useful in the diagnosis of certain diseases or treatment methods.

In a further step, the main dataset is supplied for processing by the data processing system.

Between or during the individual method steps, data compressions and/or storage operations of the captured or processed data can be provided. More particularly, the data of the calibration and the raw data of the data capture can be stored for reference purposes prior to further processing.

The data processing system is more particularly provided to identify specific events in the captured data or to identify sub-components, and to optionally filter these out of the overall dataset.

A specific event is for example the flow sound in the carotid, a breathing sound, heart sound etc. It is conceivable here that events which continue to be specified can be filtered out of the data. The flow sound of individual heart valves can thus be isolated from the recorded heart sounds, for example.

Such a splitting of the main dataset into sub-components is carried out with the aid of special filter algorithms, the use of wavelet transformation and Fourier transformation or short-time Fourier transformation (STFT), autoregressive models (AR or ARMA models) and other methods.

The splitting and evaluation of the data can also be supported by special self-learning algorithms (artificial intelligence, AI) and continuously improved.

In a further operational step, the previously broken-down sub-components can be linked with one another again and be evaluated jointly and dependent on one another using deep learning tools and big data, for example.

It is also conceivable that the collected data can be evaluated combined with patient-related data such as demographic data or comorbidities.

As is known, isolated findings or symptoms are rarely revealing and for a reliable diagnosis should always be analyzed in conjunction with other characteristics. Only in summation do individual characteristics lead to a reliable diagnosis of pathological states and permit statements to be made and provide information about an actual anomaly, a worsening of a state or an improvement in a state. This information, consisting of individual data and reference data, can then be provided to a clinician in a correspondingly prepared form.

With the proposed computer-assisted auscultation device (CAAS), a completely individualized data preparation and data analysis is thus ultimately possible. Future data recordings can then be compared not only with known other data (big data), but also with the already known individual data of the patient. As a result, diagnosis options can be based not only on the characterizing of obtained data with regard to deviations from the known or defined norm, but also specifically with regard to deviations from data collected from the same patient. This can not only permit conclusions to be drawn about developing diseases based on changes, but can also serve to check the progression of a particular therapy, etc.

If, for example, the coughing sound is different on different occasions when data is captured, this may indicate a problem. Or an assessment can be carried out as to whether the carotid flow has changed for the better or worse as a result of the intake of pharmaceuticals or other measures.

The computer-assisted auscultation device (CAAS) according to the invention therefore permits a simple and also, above all as a result of the initial data recording, reproducible data capture and, as a result of the algorithm-based data comparison, an objective quantification and qualification of body sounds.

The CAAS according to the invention thus offers significant advantages over known auscultation devices and more particularly over a stethoscope, specifically: integrated data processing, storage and management of data in databases, an objective computer-based analysis and the visualization of results. Furthermore, a subsequent documentation of the results in digital records is possible with the possibility of remote access for users.

Thus the possible uses of the inventive CAAS are not just restricted to heart or lung sounds and corresponding pathologies.

A possible evaluation and diagnosis method for pathological states based on the body's own sounds using the CAAS according to the invention could comprise the following steps:

(A) Creating a first initial dataset using the auscultation element;
(B) Transmitting the initial dataset from the auscultation element to the data processing system;
(C) Assessing the data quality of the initial dataset;
(D) Creating a main dataset using the auscultation element;
(E) Transmitting the main dataset from the auscultation element to the data processing system;
(F) Analyzing, by the data processing system, the main dataset.

It is obvious for a person skilled in the art that the method having the method steps (A) to (F) can at any time be supplemented by routine intermediate steps, which can more particularly relate to steps of data storage and data compression or format conversion.

Furthermore, for clarification of the individual method steps, reference is made to both the statements above and the statements below in connection with the description of the drawings, which can at any time supplement and clarify the described method. Reference is made here in particular to the options described there for assessing the data quality in step (C) and the options for data analysis in step (F) and the resulting diagnostic options.

In particular, reference is made to the options for assessing the data quality in step (C) by means of a comparison with known reference data or an optical check on the basis of the visualization in each case with the aid of the data processing system, and the options for data analysis in step (F), more particularly by splitting the main dataset with the aid of special filter algorithms, the use of wavelet transformation and Fourier transformation or short-time Fourier transformation (STFT), autoregressive models (AR or ARMA models) and other methods.

Not least, the use of AI algorithms and deep learning on the basis of big data within the framework of the described method can lead to further improvements in data quality as well as in analysis and diagnosis.

The invention and the technical environment are explained hereinafter on the basis of the drawings. It must be noted that the drawings show a particularly preferred embodiment of the invention. However, the invention is not restricted to the shown embodiment. More particularly, the invention comprises, as far as it makes technical sense, any combinations of the technical features which are listed in the claims or described in the description as relevant to the invention.

Shown are in:

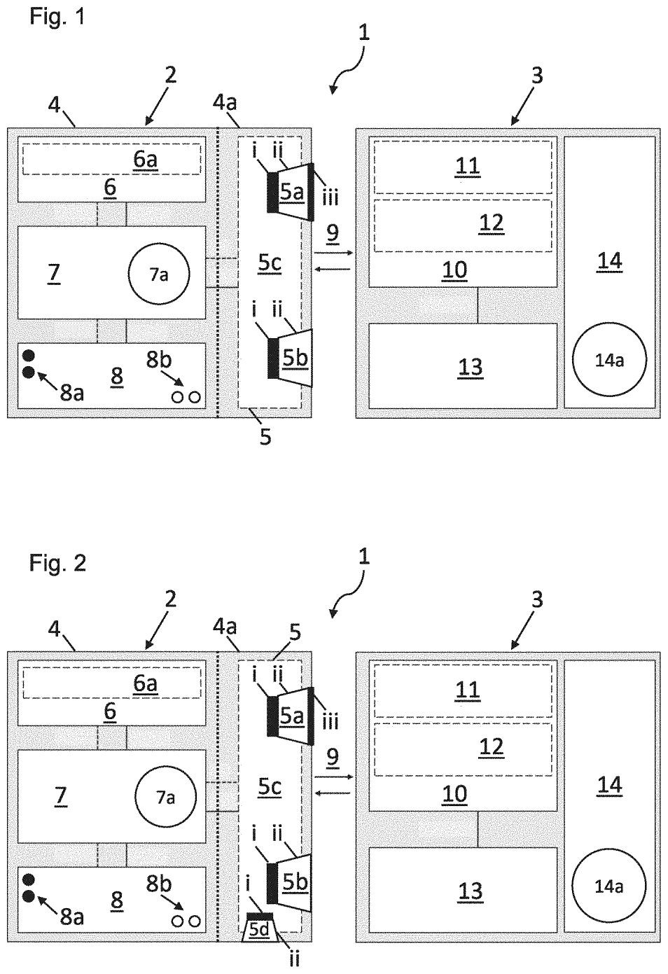

FIG. 1 a schematic representation of a first embodiment of the CAAS according to the invention,

FIG. 2 a schematic representation of a second embodiment of the CAAS according to the invention.

In a first embodiment according to FIG. 1, the CAAS 1 according to the invention comprises an auscultation element 2 and a data processing system 3. The auscultation element 2 comprises a housing 4 in which an audio system 5, a power supply 6, a computing unit 7 and a control interface 8 are arranged. Between said components, data and power connections are provided, represented by solid and dotted lines, respectively.

The audio system 5 comprises at least two microphone units 5a, 5b, which each comprise a microphone i which is connected to the housing wall 4 via a funnel-shaped connection element ii. At the connection point with the funnel-shaped connection element ii, the housing wall 4 can have openings (not shown) which are covered by a membrane iii. The audio system 5 can be provided as exchangeable with a part of the housing 4a. The microphone units 5a, 5b are preferably arranged on a board 5c which is provided for example for the digital conversion of the audio signals. Embodiments having only one microphone unit are naturally also conceivable if this suffices to capture the desired data, i.e. for example a desired frequency range can be covered.

The power supply 6 comprises a battery or a rechargeable battery 6a.

The computing unit 7 is provided for controlling the processes of the auscultation element 2. This also includes, for example, the controlling of the data transmission 9, which can be carried out in a wireless or wired manner via the means for data transmission 7a. The computing unit 7 also controls the control interface 8.

The control interface 8 is provided for operating the auscultation element 2 and for displaying the respective operating states. For this, the control element 8 comprises both operating elements 8c, preferably in the form of buttons, as well as signal generators 8b, preferably in the form of LEDs.

The signal generators 8b can be provided for displaying the operating states such as on or off of the auscultation element or to provide feedback on current processes such as, for example, the data capture or data transmission.

The data processing system 3 comprises a user interface 10, which comprises an input interface 11 and a display 12 for visualizing for example the transmitted or processed data. The data processing system 3 further comprises a local memory 13, which can comprise a database. The data processing system 3 naturally further comprises a computing unit 14 for analyzing data, for controlling the wireless or wired data exchange via the means for data transmission 14a etc.

For switching on the auscultation element 2, the power supply 6 is activated for example by using a rechargeable battery 6a or by means of an input via the control interface 8. After the start-up operation, an automatic connection between the auscultation element 2 and the data processing system or a Wi-Fi network can be established. A successful connection can be confirmed via the control interface 8.

The auscultation element 2 is now ready to start the auscultation process, and can be controlled for example via a switch 8c. It is thus conceivable that the initial data capture or calibration is started using a first switch 8c. The achieved data quality can be displayed to the user for example via an LED 8b. Sufficient data quality could be displayed via a green LED, insufficient data quality via a red LED, for example. This enables the user to find an appropriate position of the auscultation element 2 in order to ensure sufficient data quality or high signal quality and amplitude.

As soon as sufficient data quality has been confirmed, the actual data capture can be started in a second step via a switch 8c and be completed after a sufficient duration. The status of the data capture can in turn be displayed via an LED 8c.

The captured data can be transmitted to the data processing system 3 either in real time or after caching in the auscultation element 2, as desired, and can optionally be stored in the data processing system prior to a further evaluation and/or visualization.

After data capture has been completed, the auscultation element 2 can be switched off via a switch 8c on the control interface 8.

The data capture system 3 serves to evaluate and visualize the data captured by the auscultation element 2. It also enables patient-related data, such as demographic information or comorbidities, to be added and captured data to be organized and accessed.

Visualization preferably starts on data transmission from the auscultation element 2 and stops as soon as data transmission has come to an end. This enables the user to perform a real-time interpretation of the auscultation signal and optionally to find a suitable capture location in order to ensure sufficient signal quality without necessarily needing a computer-assisted data comparison with reference data.

The second embodiment shown in FIG. 2 differs from the first embodiment of the CAAS 1 according to the invention shown in FIG. 1 in that the auscultation element 2 comprises a further microphone unit 5d which is preferably provided for capturing external sounds, i.e. mainly not the body's own sounds, or can be used for recording the external portion of coughing sounds. The further microphone unit 5d can preferably be provided as part of the exchangeable housing part 4a and be arranged on the same board 5c of the audio system 5 as the other microphone units 5a, 5b. However, it is also conceivable that the further microphone unit 5d is provided at another place on the housing 4, particularly because to record external sounds, said microphone unit 5d is not necessarily in contact with the patient's body, and therefore less stringent hygiene requirements might apply and an exchange does not necessarily need to be provided for.

More than just the one microphone unit 5d shown here by way of example can naturally be provided for the data capture of external sounds.

The data recorded by the further microphone unit 5d can be included in the analysis of the body sounds captured by the microphone units 5a, 5b and can be used for example to filter corresponding external sounds out of the dataset of the body's own sounds. This is advantageous for the clarification of and delimitation between the body's own sounds and exogenous (or external) sounds.

### LIST OF REFERENCE CHARACTERS

1 Auscultation device (CAAS)
2 Auscultation element
3 Data processing system
4 Housing (4a: separable housing part)
5 Audio system (5a, 5b, 5d: microphone unit; 5c: board; i: microphone; ii: connection element; iii: membrane)
6 Power supply (6a: battery/rechargeable battery)
7 Computing unit (7a: means for data transmission)
8 Control interface (8a: operating element/button; 8b: signal generator/LED)
9 Data transmission
10 User interface
11 Input interface

### 12 Display

### 13 Memory

14 Computing unit (14a: means for data transmission)

1. An auscultation device for capturing and evaluating body sounds, comprising:
an auscultation element having a housing and arranged therein: an audio system for capturing sounds, a power supply, a computing unit, means for data transmission and a control interface; and
a data processing system having a user interface, a memory, a computing unit for analyzing sounds and means for data transmission,
wherein the audio system (5) comprises at least one microphone unit for capturing the body's own sounds and this at least one microphone unit comprises a microphone and a connection element, wherein the connection element is provided between microphone and housing.

2. Auscultation device according to claim 1, wherein at least one of the microphone units comprises a membrane which rests against the housing.

3. Auscultation device according to claim 1, wherein the connection element is funnel-shaped.

4. Auscultation device according to claim 1, wherein the data transmission relates to the data captured by the audio system.

5. Auscultation device according to claim 1, wherein the data processing system comprises a display for visualizing the transmitted data.

6. A method for the diagnosis of pathological states using an auscultation device according to claim 1, said method comprising the following steps:
(A) Creating an initial dataset using the auscultation element;
(B) Transmitting the initial dataset from the auscultation element to the data processing system;
(C) Assessing the data quality of the initial dataset;
(D) Creating a main dataset using the auscultation element;
(E) Transmitting the main dataset from the auscultation element to the data processing system;
(F) Analyzing, by the data processing system, the main dataset.

7. Method according to claim 6, wherein the initial dataset comprises at least two heart impulses, a swallowing sound, the blood flow of the carotid, a cough and a breath.

8. Method according to claim 6, wherein the assessment of the data quality is carried out by means of a comparison with reference data.

9. Method according to claim 6, wherein the analysis of the main dataset is carried out with the aid of filter algorithms, the use of wavelet transformation, Fourier transformation, short-time Fourier transformation (STFT) or autoregressive models (AR or ARMA models).

10. Method according to claim 9, wherein the analysis of the main dataset is further carried out via the use of AI, big data and deep learning tools.
